(19) United States
(12) Patent Application Publication
(10) Pub. No.: US 2011/0294897 A1
(43) Pub. Date: Dec. 1, 2011

(54) Treating Xerophthalmia With Compounds Increasing Meibomian Gland Secretion

(75) Inventors: A.K. Gunnar Aberg, Sarasota, FL (US); Vincent B. Ciofalo, Branford, CT (US)

(73) Assignee: Bridge Pharma, Inc., Sarasota, FL (US)

(21) Appl. No.: 12/956,386

(22) Filed: Nov. 30, 2010

Related U.S. Application Data
(60) Provisional application No. 61/283,327, filed on Dec. 2, 2009.
(60) Provisional application No. 61/343,258, filed on Apr. 26, 2010.

(51) Int. Cl. A61K 31/137; A61P 27/04

(52) U.S. Cl. 514/653

(57) ABSTRACT

Methods for the treatment of patients suffering from dry eyes, by using adrenergic beta-receptor agonists, particularly salbutamol and in particular the optically pure or substantially pure R-enantiomer thereof. The embodiments disclosed herein include methods of increasing the Meibomian lipid secretion and thereby reducing or eliminating xerophthalmia symptoms by the administration of formulations containing therapeutically effective amounts of an adrenergic beta-receptor agonist to said patients. In particular, certain embodiments disclosed herein concern compositions that contain R-salbutamol as the active beta-receptor stimulating ingredient. In certain embodiments, the formulations are administered to the ocular surface of the eye and/or to the eyelid (the underside of the eyelid and/or the top of the eyelid) of a patient in need thereof.

This application claims priority of provisional application Ser. No. 61/283,327 filed Dec. 2, 2009, and Ser. No. 61/343,258, filed Apr. 26, 2010, the disclosures of which are hereby incorporated by reference.

### FIELD

The present disclosure concerns the medicinal treatment of xerophthalmia, also called dry-eye syndrome, using adrenergic beta-receptor agonists, particularly salbutamol and preferentially the R-isomer of salbutamol to increase Meibomian gland secretion.

### BACKGROUND

Xerophthalmia is an eye disease in mammals and in particular in humans and dogs, which causes may include decreased lacrimal and Meibomian gland secretion and/or increased evaporation from the eye and/or contact lens intolerance. This disease is alternatively called, for example “dry eye disease” or “dry eye syndrome” or “dry eyes” or “xerophthalmia” or “xerophthalmic disorder” or “keratoconjunctivitis sicca” or “hypolacrimia”. Other names exist and as the knowledge of the pathophysiology of dry eye disease expands, the associated terminology continues to evolve. Although the terms may represent various forms of this disorder, the terms are used interchangeably herein and are considered as synonyms in this document. All forms of dry eye disease result in dehydration and consequential tear hyperosmolarity (Lemp 1995, which publication is hereby included by reference.) The symptoms of xerophthalmia may vary between patients, but include one or more symptoms, such as for example ocular dryness, ocular burning, sandy-gritty eye irritation, ocular foreign-body sensation and/or photophobia (Keratoconjunctivitis Sicca. Wikipedia, November 2010; which publication is hereby incorporated by reference.) The disease may be so severe that it leads to corneal scarring and blindness.

The hydrating and lubricating tear film covering the eye is generally considered as containing three different layers: a mucous, an aqueous layer and a lipid layer. The mucous layer originates from goblet cells in the mucous membranes surrounding the eye. The aqueous component is secreted from the lacrimal glands and the lipid components are secreted from the Meibomian glands. The lipid layer is the most distal layer from the epithelium. The lipids offer lubrication and most importantly inhibit excess evaporation of water from the tear film. The development of dry eye disease can arise due to various pathological conditions, such as for example lacrimal gland deficiency, Meibomian gland deficiency, vitamin deficiency, allergies, drugs, and hormonal changes. Dry eye syndrome can be of two different types: Evaporative Dry Eye (EDE) disease and Aqueous Deficient Dry Eye (ADDE) disease based on said differences in etiopathogenesis. Thus, EDE results from Meibomian gland dysfunction and ADDE is the result of lacrimal gland dysfunction. Dry eye disease may in some patients include symptoms of both diseases (EDE and ADDE.) Those skilled in the art of ophthalmology will avoid using drugs that increase lacrimal secretion in patients suffering from EDE since existing lipids will be washed away with the increased tear flow, as is known to happen during crying.

EDE is usually caused by decreased Meibomian gland secretion and is one of the most common clinical presentations for ophthalmologists and is often expressed as plugged or capped Meibomian glands or glands producing a foamy tear film. The secretion from the Meibomian glands of patients suffering from EDE may have high viscosity, looking like toothpaste. The current therapy for patients with Meibomian gland dysfunction includes warm compresses and eyelid massage (Bowling et al. 2010, which publication is hereby incorporated by reference.) No medication exists that increases Meibomian gland secretion. It is now believed that stimulation of the natural secretion of the Meibomian glands will help prevent these glands from getting plugged and also will reduce the incidence of Meibomitis.

R-salbutamol is the chemically and optically pure R(−)-isomer of a′[(tert-butylamino) methyl]-4-hydroxy-m-xylene-a,a′-diol, and any biologically acceptable salt thereof. Other chemical names of this compound exist. The term “salbutamol” most often refers to the free base, or a salt thereof, such as for example the hydrochloride, the hemisulfate (sometimes called “sulfate”) or the tartrate salt.

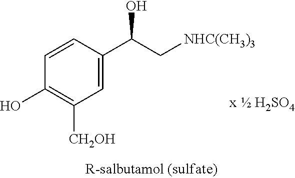

Salbutamol and R(−)-salbutamol sulfate have the molecular formula C₁₃H₂₁NO₃.½H₂SO₄ and the molecular weight is 288.31. Most salts, such as for example the sulfate, hydrochloride and tartrate salts of salbutamol and R(−)-salbutamol are white odorless, crystalline powders that are readily soluble in water.

Polymorphs of R-salbutamol have been described by Hamied et al. in WO 02/48090 A1 (which patent application is hereby included by reference) and additional polymorphs may exist. All polymorphs are included within scope of the embodiments disclosed herein.

The racemic compound RS-salbutamol (salbutamol) is a well-known medication for asthma in human patients and is sold under various trade names and generic names, such as for example Proventil® (Schering), Ventolin® (Glaxo) and Albuterol (Cipla). R(−)-salbutamol is also an asthma medication, sold under the trade names Levolin® (Cipla) and Xopenex® (Sepracor). The use of R(−)-salbutamol to treat asthma in humans was originally described by Barberich et al. in U.S. Pat. No. 5,362,755. The use of R(−)-salbutamol to inhibit premature contractions (tocolysis) of the pregnant uterus in humans was described by Pesterfield in U.S. Pat. No. 5,708,036, the use of R(−)-salbutamol as a growth promoter in livestock was described by Aberg et al. in U.S. Pat. No. 6,110,974, and the use of R-salbutamol for the treatment of heaves in horses was described by Ciofalo in US Patent Application 20050020692.

Pharmacologically, both salbutamol (called albuterol in the USA) and the R-isomer thereof (R-salbutamol, also called R-albuterol or levosalbutamol or levoalbuterol) are combined adrenergic beta-1 and adrenergic beta-2 receptor agonists, most known for their ability to induce relaxation of bronchial smooth muscles (Salbutamol, Wikipedia, Nov. 17, 2010; Levosalbutamol, Wikipedia, Nov. 1, 2010). The compounds salbutamol and R-salbutamol have practically no affinity for adrenergic beta-3 receptors (Example 4). Some beta-receptor agonists, such as for example terbutaline and fenoterol, have been described as having additional beta-3 agonistic effects (Horinouchi et al, 2001, which publication is hereby incorporated by reference). Terbutaline, fenoterol and other compounds and optically active isomers thereof, with adrenergic beta-1, beta-2 and/or beta-3 agonist activities, or any combinations thereof are included within the scope of the embodiments disclosed herein. Salbutamol and R-salbutamol are not selective beta-receptor agonists, but have also beta-receptor antagonistic (“beta-blocking”) activity and are therefore partial agonists (Penn et al., 1996, which publication is hereby incorporated by reference.) All compounds with beta-1, beta-2 and/or beta-3 adrenergic agonistic activities are included within the scope of the embodiments disclosed herein.

Methods of making R(−)-salbutamol have been described by Hamied in WO 02/48090 A1, by Gao in U.S. Pat. No. 5,399,765 and by Ferrayoli et al., 2000, which documents are hereby incorporated by reference. R-salbutamol is commercially available from Cipla Pharmaceuticals, Mumbai, India and from Sepracor, Marlborough, Mass., USA.

The overall prevalence of dry eyes was found to be 14.4% in a cohort aged 48 to 91 years (Moss et al., 2000; which publication is hereby incorporated by reference). It has also been estimated that one in four patients consulting ophthalmologists complain of dry eyes and up to 20% of adults aged 45 years and older experience dry eye symptoms (Brewitt et al., 2001, abstract; which abstract is hereby incorporated by reference).

Contact lenses provide a valuable option to the vision impaired. Although contact lenses have been much improved, ocular irritation is still a common problem and wearers often experience symptoms of dry eyes due to moisture loss from the contact lenses (Bowling, 2007; which publication is hereby incorporated by reference). Additionally, contact lenses rest on the tear film and if absent, the lenses rest on the cornea, causing discomfort, pain and possibly corneal damage.

Dry eye disease and the symptoms thereof are vastly different from allergic conjunctivitis and similar inflammatory diseases. The symptoms for dry eye disease include dryness, burning, sandy-gritty eye irritation, foreign-body sensation, photophobia (Keratoconjunctivitis sicca. Wikipedia, November, 2010; which publication is hereby incorporated by reference.) The symptoms for allergic conjunctivitis include red eyes and itching, which symptoms are associated with allergies and are related to histamine and other inflammatory mediators (Reid, 2006, which publication is hereby incorporated by reference.) Individual patients may simultaneously suffer from both dry eyes and conjunctivitis and possibly other ocular disorders. A large number of patients suffer from a combination of seasonal allergic conjunctivitis and dry eyes and their conditions—usually described as “sandy-gritty eyes”—can be predicted as they are often correlated with pollen seasons, which make these patients as risk for developing dry eyes syndromes. These patients will benefit from pretreatment with the drugs disclosed herein, which, with high likelihood, will prevent the suffering of these patients from seasonal dry eye symptoms.

### Lacrimal Glands, Accessory Lacrimal Glands and Meibomian Glands

The glands contributing hydration and lubrication of the mucous membranes around the eye are described in numerous textbooks in ophthalmology, such as for example Beuerman et al., 2004 and Mathers 2004, which publications are hereby incorporated by reference. It is known that adrenergic beta-receptor agonists may increase lacrimal flow after systemic administration (Aberg et al., 1979 and U.S. Pat. No. 6,569,903, which publications are hereby incorporated by reference). To our knowledge, increased Meibomian secretion by compounds with beta-adrenergic stimulatory activities has previously not been shown or described. Actually, we are not aware of any pharmacologically induced stimulation of Meibomian glands in vivo previously being shown or described. In particular, to our knowledge, no drug has previously been shown to demonstrate improved or increased expression of Meibomian gland secretion in vivo after ocular administration.

There are two types of lacrimal glands: The Main Lacrimal Glands and The Accessory Lacrimal Glands. The main lacrimal glands are anatomically located at some distance from the eye and cannot be directly stimulated by drugs that are applied to the eye. The accessory lacrimal glands are located in the in the mucus membranes on the eye and surrounding the eye and can be reached after topical ocular drug administration to the eye or the membranes surrounding the eye, such as for example instillation into the conjunctival sac.

As previously pointed out, the Meibomian glands are anatomically located in the eyelids and are secreting lipids that have lubricating activity on the mucous membranes of the eye. Importantly, the lipids from the Meibomian glands also form the outer layer of the tear film, protecting the watery component of the tear film from evaporating (Mathers, 2004, which publication is hereby incorporated by reference.) Thus lipid secretion from the Meibomian glands has two different and important functions in the eye: lubrication of the mucous membranes and inhibition of tear film evaporation.

### Current Treatment of Dry Eye Disease

Current treatments of dry eye disease were reviewed by Gayton, 2009, which publication is hereby incorporated by reference. It was pointed out that artificial tears offer only a temporary palliative effect, while corticosteroids are effective disease-modifying agents for patients suffering from dry eye disorders. However, topical corticosteroids are not approved as treatment for dry eyes in the US and other countries and are not recommended for long-term use because of the known risks for significant adverse effects in the eye, which include increased intraocular pressure and/or the development of cataracts.

The only drug that is presently approved in the US for the treatment of dry eyes is cyclosporin (Restasis®, Allergan), which is a potent immunosuppressive drug. Cyclosporin, which is the active ingredient in Restasis®, is a large molecule with a molecular weight of more than 1200 daltons, which probably is the main reason why cyclosporin penetrates tissues with difficulty, if the molecule is able to penetrate the ocular tissues at all. Thus, it is not believed and it has not been shown that cyclosporin is able to penetrate the ocular tissues to reach the main lacrimal glands or the Meibomian glands. The therapeutic effects of Restasis® have slow onset and full activity may be obtained only after twice daily use of the drug for up to 6 months.

The manufacturer cites four clinical studies performed in approximately 1200 patients with moderate to severe dry eyes. A total of 15 percent of Restasis®-treated patients experienced an improvement in ocular wetness, as determined by Schirmer test scores of 10 mm or greater. The most common side effect following the use of Restasis® is ocular burning, which according to the manufacturer occurred in 17 percent of Restasis®-treated patients (Physicians' Desk Reference, 2009, p. 557; which page is hereby incorporated by reference). Since cyclosporin is a potent immunosuppressive drug, and in light of the limited therapeutic success of the drug, ophthalmologists may not want to use this drug when ocular infections are present, which are common in patients suffering from dry eyes.

### Pharmacologically Known Effects of Adrenergic Beta-Receptor Agonists on Lacrimal Secretion

In the original publication by Aberg et al. (1979), it was pointed out that the systemic effects of the non-selective adrenergic beta-receptor agonist isoprenaline on lacrimal secretion most likely are due to adrenergic beta-1 stimulation. Stimulatory effects by selective adrenergic beta-2 agonists on lacrimal secretion were described by Honma et al. (U.S. Pat. No. 6,569,903), and increased lacrimal secretion by adrenergic beta-3 receptor activation was described by Horinouchi et al., 2001 and by Kobayashi et al. in US Pat Appln 20080306160, which documents are hereby incorporated by reference. Those skilled in the art of ocular pharmacology are also aware that lacrimal gland secretion can be achieved by endogenous cholinergic stimulation (epinephrine or norepinephrine), or by drugs having muscarinic effects, for example by pilocarpin or carbachol. Lacrimal gland stimulation can also be caused by emotions (crying) or by ocular irritation.

Lacrimal stimulation in itself does not offer relief to patients who are suffering from dry eye syndrome, since increased lacrimal secretion may flush out the lipids that protect the tear film from evaporation. Thus, many dry eye sufferers experience watery eyes since the lacrimal glands overcompensate for the irritation caused by an abnormal tear film (Dobson, 2001). Thus, compounds that solely increase lacrimal tear flow are not useful as remedies for patients suffering from dry eye syndromes.

As known by those skilled in ophthalmology, the watery lacrimal secretion will rapidly evaporate if not protected by the lipids, which are secreted from the Meibomian gland and which form the lipid outer layer of the normal tear film. To our knowledge no drugs have previously been demonstrated to stimulate Meibomian lipid secretion in vivo. It has now surprisingly been found that adrenergic beta-receptor agonists in general and the non-inflammatory and non-irritating adrenergic beta-2 receptor agonist R-salbutamol in particular will increase both Meibomian gland secretion (Examples 6 and 7) and lacrimal gland secretion (Example 5), which will be a beneficial combination of effects for patients suffering from all types of dry eye disease.

### Side Effects of Adrenergic Beta-2 Receptor Agonists

Since drugs with anti-inflammatory effects, such as steroids and cyclosporin have therapeutic activity against dry eye syndromes, it is obvious that drugs with pro-inflammatory activity should be avoided by patients suffering from dry eyes or by individuals at risk for developing dry eye disease. Individuals at risk for developing dry eye disease are for example persons with a history of seasonal allergic “sandy-gritty” syndromes. While considering an adrenergic beta-receptor agonist for ocular use in patients with dry eye disease or who are at risk of developing dry eye disease, it is important that a drug without pro-inflammatory activity is selected. While most beta-adrenergic agonists have pro-inflammatory activities, R-salbutamol is free from pro-inflammatory activity since the pro-inflammatory effects of racemic salbutamol reside solely in the S-isomer, as shown or described in numerous publications (Baramki et al., 2002, Agrawal et al., 2004, Henderson et al., 2005, Volcheck et al., 2005).

Racemic salbutamol is also known to cause other side effects, as for example myotropic hyperreactivity or hyperactivity. Said hyperreactivity will be avoided by using the single R-isomer of salbutamol rather than racemic salbutamol, since R-salbutamol does not cause said types of muscle hyperactivities (Andersson et al. 1996 (Abstract), Johansson et al. 1996, Agrawal et al., 2004, Henderson et al., 2005.) As known by those skilled in the art, the muscle of Riolan, when activated may mechanically constrict the orifices of the Meibomian glands, thereby decreasing the secretion of the lipids from said glands. Hyperreactivity of the muscle of Riolan has not been described as a side effect of beta-adrenergic drugs, but taking the risk for such effects into consideration may be appropriate since the muscle of Riolan is located in the distal area of the eyelids, close to the secretory ducts from the Meibomian glands.

Adrenergic beta-agonists may cause systemic side effect, as is well known to those skilled in the art of pharmacology. Thus, the stimulation of either beta-1 receptors or cardiac beta-2 receptors in the heart is dose-dependent and will occur at plasma concentrations that are significantly higher than those obtained from the relatively low doses instilled in the eye according to the embodiments disclosed herein. Likewise, the risk for other systemic side effects, such as for example tremor, are remote due to the low doses of beta-receptor agonists that are placed in the eyes according to the embodiments disclosed herein, as it should be kept in mind that most of the fluids from the eye are drained from the eyes to the nose through the nasolacrimal ducts and will therefore not reach the systemic circulation.

### SUMMARY

This disclosure also relates to compositions containing adrenergic beta-receptor agonists and particularly the optically pure or substantially pure beta-receptor agonist R(−)-salbutamol, for use by patients suffering from dry eyes, and method of administration thereof. The method and compositions presented herein, offer potent, long-lasting therapeutic activity in patients suffering from dry eyes, while avoiding or reducing adverse effects including but not limited to pro-inflammatory activity and smooth muscle hyperreactivity.

The embodiments disclosed herein include novel methods for the treatment of patients suffering from dry eyes, by using adrenergic beta-receptor agonists, particularly salbutamol and in particular the optically pure or substantially pure R-enantiomer thereof. It has now been found that compounds with adrenergic beta-receptor agonistic activity will express increased Meibomian gland secretion in addition to the previously described increase of lacrimal secretion. It is a well accepted fact that just increasing the amount of the watery tears from the lacrimal glands is of no or very limited therapeutic importance for patients suffering from dry eye disease. Likewise, crying, which increases lacrimal tear secretion, is not of therapeutic value, but may actually have the opposite effect since the increased tear flow washes out Meibomian lipids, thereby aggravating the situation. Adrenergic beta-receptor agonists, preferably R-salbutamol, can be administered by ocular instillation, which will prevent, decrease and/or limit the prevalence and severity of systemic side effects, such as for example tachycardia, tremors or metabolic side effects. Adrenergic beta-receptor agonists can also be administered by the intra-nasal route, such as for example by nasal insufflation or by nasal drops, or by the use of devices such as metered dose inhalers, nebulizer dry powder for inhalation or insufflation. A formulation containing a therapeutically amount of an adrenergic beta-agonists can also be applied on the eyelids, which may be in addition to or instead of a topical/ocular administration. Adrenergic beta-agonists can be administered orally in the form of, for example, tablets, capsules or syrups. Tablets and capsules may be of instant or controlled release types. Adrenergic beta-agonists can also be administered to the patient by means of devices that release the drug over time, after being applied to the eye or the mucous membranes surrounding the eye. The solutions described herein or modifications thereof, may be used for nasal drop or spray administration. To our knowledge, selective beta-adrenergic agonists have never been used therapeutically in ophthalmology.

The embodiments disclosed herein include methods of increasing the Meibomian lipid secretion and thereby reducing or eliminating xerophthalmia symptoms by the administration of formulations containing therapeutically effective amounts of an adrenergic beta-receptor agonist to said patients. In particular, certain embodiments disclosed herein concern compositions that contain R-salbutamol as the active beta-receptor stimulating ingredient. In certain embodiments, the formulations are administered to the ocular surface of the eye and/or to the eyelid (the underside of the eyelid and/or the top of the eyelid) of a patient in need thereof.

The effects of R-salbutamol on lacrimal and Meibomian secretion, as well as pharmacological, physicochemical and pharmaceutical properties of the compound have been studied. All tested adrenergic beta-receptor agonists were found to increase lacrimal tear secretion after systemic administration. This systemic effect is believed to be the result of simulation of the main lacrimal glands, but because of the anatomical location of said main lacrimal glands, drug may not reach these glands after instillation into the eye. However, drugs will reach the accessory lacrimal glands after local instillation to the eye or into the conjunctival sac.

The Meibomian secretion consists of lipids and the lipid film is of pivotal importance since said lipids lubricate the mucous tissues and decrease the evaporation of water from the ocular tissues. The Meibomian glands, which are located in the eyelids, normally have 30-40 orifices on the rim of each eyelid. Anatomically, the Meibomian glands are located in close proximity to the fluids surrounding the eye and results from our studies (Example 7) demonstrate that small molecules like R-salbutamol can penetrate across the inner membranes of the eyelids to reach the acrinar cell structures of the Meibomian glands and increase the Meibomian secretion.

### DETAILED DESCRIPTION

It has now been found that R-salbutamol will increase not only lacrimal secretion (measured with Schirmer methodology), but surprisingly also Meibomian secretion. Meibomian gland secretion was measured with a Meibometer (Courage-Khazaka Electronic GmbH, 50829 Cologne) and was increased in vivo as described in Examples 6 and 7, hereinafter. In addition to the combined beta-1 and beta-2 partial agonist R-salbutamol, other adrenergic beta-receptor agonists, such as for example RR/SR ractopamine, also have the ability to increase Meibomian gland secretion in vivo.

The chemical purity of all batches of all compounds used herein for biological studies have been >98%. The optical purity of the single isomers used herein have optical purity >98%. The optical rotation of the R(−)-salbutamol salts ([α]²⁰_{d}) was −32 to −36. All samples of R-salbutamol used herein were of GMP-grade and were supplied by Dr. Yusuf K. Hamied, Cipla Pharmaceuticals, Mumbai, India. Racemic salbutamol were purchased from Sigma and had chemical purity of >98%.

Topical ocular formulations of R-salbutamol or a salt thereof preferably contain R-salbutamol in concentrations between about 0.001 percent and about 15 percent (calculated as base), more preferably between about 0.05 percent and about 3 percent (calculated as base), and most preferred between about 0.10 percent and about 2 percent (calculated as base). As is the case with most ocular drugs that are intended for topical/ocular administration, formulations of adrenergic beta-receptor agonists preferably have acidity preferably between about pH 4 and about pH 7 and more preferably between about pH 4.6 to about pH 6.5, which are the ranges tolerated by the eye. The preferred osmolality is between 100 mOsm and 1000 mOsm, more preferred between 150 mOsm and 450 mOsm, most preferably between 230 mOsm and 330 mOsm, which are the ranges tolerated by the eye.

In certain embodiments, methods of reducing symptoms associated with dry eye are provided, said methods comprising the administration of a formulation containing an adrenergic beta-receptor agonist, particularly salbutamol and preferentially the R-isomer of salbutamol, or a combination of an adrenergic beta-receptor agonist, particularly salbutamol and preferentially the R-isomer of salbutamol with an immunoinhibitor, such as for example cyclosporin, or an anti-inflammatory compound, such as for example norketotifen, or an antipruritic antihistamine, such as for example ketotifen, levacobastine or olopatadine. In certain embodiments, the adrenergic beta-receptor agonist(s) are administered in an amount effective to stimulate Meibomian gland secretion in an individual in need thereof. In certain embodiments, the administration of the adrenergic beta-receptor agonist(s) stimulates the Meibomian gland secretion in an amount sufficient to relieve the symptoms of dry eye. In certain embodiments, the administration is topical, and is applied to the ocular surface of the eye.

Distomeric isomers of adrenergic beta-receptor agonists—such as for example S-salbutamol—may also increase Meibomian gland secretion, which possibly—but not necessarily—is due to optical impurities of the corresponding eutomeric isomer(s) in the samples tested.

In certain embodiments, methods of decreasing contact lens intolerance or reducing the dry eye symptoms thereof are provided, said methods comprising the administration of a formulation containing an adrenergic beta-receptor agonist, particularly salbutamol and preferentially the R-isomer of salbutamol, or a combination of said adrenergic beta-receptor agonist with an immunoinhibitor, such as for example cyclosporin, or anti-inflammatory compound, such as for example norketotifen, or another compound such as for example ketotifen, levocabastine or olopatadine.

As mentioned above, in certain embodiments, the ocular formulations may comprise a therapeutically effective amount of adrenergic beta-receptor agonists other than R-salbutamol, which may have beneficial effects for patients suffering from dry eye syndrome. Examples of other adrenergic beta-receptor agonists are racemic salbutamol, and racemic and isomeric forms of terbutaline, formoterol, salmeterol, ractopamine, fenoterol, procaterol, hexoprenaline, pirbuterol, mabuterol, banbuterol, formoterol, epinephrine, isoprenaline, ractopamine and tulobuterol, which are all included in the embodiments disclosed herein. Likewise, adrenergic beta-3 agonists or eutomeric isomers thereof may increase Meibomian secretion and be of therapeutic value as medication for patients suffering from dry eye disease. Some adrenergic agonists, such as for example epinephrine can be replaced by prodrugs of said agonists, such as for example dipivefrin, which is an ester prodrug, of epinephrine and pivalic acid that is hydrolysed to form epinephrine. The use of prodrugs of adrenergic beta-receptor agonists may be preferred since prodrugs may have less side effects than the parent drugs or may penetrate the tissues to reach the biophase more easily than the parent drug(s). Thus, according to the manufacturer, dipivefrin causes epinephrine intolerance in only 3% of the patients, while epinephrine causes said type of intolerance in 55% of the patients (Propine. PDR for Ophthalmic Medicines. 2007), which article is hereby incorporated by reference. All prodrugs to adrenergic beta-receptor agonists—including prodrugs of R-salbutamol—are included in the embodiments disclosed herein. Some adrenergic beta-receptor agonists, such as for example formoterol and ractopamine have two chiral centers and four isomers, all of which isomers, and combinations thereof, are included in the embodiments disclosed herein.

Various diseases and circumstances may result in dry eyes and examples are keratoconjunctivitis sicca, age-related dry eye, contact lens intolerance, Stevens-Johnson syndrome, Sjögren's syndrome, ocular cicatrical pemphigoid, blepharitis, corneal injury, infection, Riley-Day syndrome, congenital alacrima, nutritional disorders or deficiencies (including vitamin A deficiency), atopic, autoimmune and other immunodeficient disorder, and side effects of medications. The methods of reducing the symptoms of dry eye disease, disclosed herein are useful, regardless of the etiology of the dry eye syndrome being treated.

In certain embodiments, it is determined if a patient is suffering from a xerophthalmic disorder, and if said determination is positive, an ophthalmic composition comprising a therapeutically effective amount of an adrenergic beta-receptor agonist, particularly salbutamol and preferentially the R-isomer of salbutamol, or a pharmaceutically acceptable salt thereof is administered to said patient in an amount and a concentration that is sufficient to achieve therapeutic effects of said compound(s) in said patient. Said diagnosis of xerophthalmia can be performed by a qualified physician, using interviews, physical examination and/or application of a standardized test, such as for example Schirmer's test and fluorescein tests of tear film break-up time. Reviews of the diagnosis of kerato-conjunctivitis sicca can be found in The Merck Manual, 18th Ed. 2006) and in Wikipedia, November 2010. Both documents are hereby incorporated by reference. Methods for diagnosis of dry eyes can also be found in textbooks in ophthalmology such as for example Fechner et al. 1997, pages 359-360, which pages are hereby incorporated by reference.

Improved Meibomian secretion by the use of an adrenergic agonist, preferably R-salbutamol, has additional beneficial effects since obstructions of the Meibomian ducts are less likely to form and blepharitis will be less likely to develop as a consequence of the improved secretion through the ducts. Obstruction by keratinization of the Meibomian gland ducts may also become less likely as a consequence of testoid pharmacological activities of certain adrenergic agonists, such as for example salbutamol. The preventive use of an adrenergic beta-receptor agonist, particularly R-salbutamol, may therefore prove to be of significant value to patients who are prone to develop blepharitis. Thus, it is expected that clinical tests will demonstrate blepharitis to be an additional indication for R-salbutamol. In general, patient should not stop using medication for dry eye disease as soon as the therapeutic goal has been reached, but shall continue with the medication to prevent recurrence of the symptoms—an example is a patient with seasonal dry eye symptoms, who need to take the medication during the pollen season to prevent the disease to recur.

### DEFINITIONS

The terms “tear” and “tears”, as used herein and in most scientific publications and previous patents refer to the watery lacrimal secretion that is measured by Schirmer methodology.

The term “tear film” as used herein refers to the three layer of protection (mucous layer, watery layer and lipid layer) that have been described herein.

The term “tear film break up time” as used herein, refers to the time required for the ocular surface to lose cohesive surface wetting after each blink.

The terms “about” and “approximately” where used in this document refer to ±10 percent. Thus, as examples “about 10 percent” refers to “from 9 percent to 11 percent” and “approximately pH 6” refers to “from pH 5.4 to pH 6.6”.

The term “adrenergic beta-receptor agonist”, “beta-receptor agonist”, etc. refer to compounds that have affinity for adrenergic beta-receptors and activate said receptors.

The term “partial agonist,” means that a compound has both agonistic and antagonistic activity.

The terms “disorder” and “disease” are used as synonyms herein.

The terms “patients” and “subjects” in this document refer to mammals, primarily humans, dogs and cats and are used as synonyms herein.

The term “optically pure” or “substantially pure” or “substantially free from” (corresponding isomers) refers to a mixture consisting of at least 90 percent of the eutomer and 10 percent or less of the distomer, preferably at least 95 percent of the eutomer and 5 percent or less of the distomer, most preferred is a mixture consisting of at least 98 percent of the eutomer and 2 percent or less of the distomer.

The term “chemically pure” refers to a compound consisting of at least 90 percent of the active moiety and 10 percent or less of chemical impurities, preferably at least 95 percent of the active moiety and 5 percent or less of chemical impurities, and most preferred is a compound consisting of 98 percent of the active moiety and 2 percent or less of chemical impurities.

The term “therapeutically effective” (amount, concentration or dose) refers to an amount, concentration or dose that yields therapeutic benefit to a patient, which in the present case refers to therapeutic benefit to a patient suffering from a xerophthalmic disorder. The actual amount of R-salbutamol yielding therapeutic benefit to a patient, suffering from xerophthalmia, depends on many factors and varies for example with the concentration of R-salbutamol in the formulation, the frequency of drug administrations, the length of time of the treatment, the administration form and the severity of the disease.

The term “salbutamol”, as used herein, refers to racemic salbutamol containing a mixture of about 50 percent of the R-isomer and about 50 percent of the S-isomer of salbutamol.

The terms “R-salbutamol”, “S-salbutamol”, “isomer” or “enantiomer” in this document refer to a single isomer, substantially free from the corresponding distomeric isomer.

The term “R-salbutamol” refers to the R-isomer of the racemic drug salbutamol and as used herein, the term “R-salbutamol” refers either to the free base or to a pharmaceutically acceptable salt form or solvate thereof.

The term “ketotifen” as used herein, most often refers to a salt thereof, such as the for example the hydrochloride or the most preferred salt form of ketotifen, which is the hydrogen fumarate salt.

The terms “eutomer”, “eutomeric”, etc. refer to one or more chiral enantiomer(s) having biologic activity.

The terms “distomer”, “distomeric”, etc. refer to one or more chiral enantiomer(s) having no therapeutic activity or less therapeutic activity than the corresponding eutomer.

The term “pharmaceutically acceptable salt” and the like refer to salts prepared from pharmaceutically acceptable acids, such as for example hydrochloric, tartaric, hydrobromic, maleic, sulphuric and fumaric acids. They are generally safe for administering to patients according to established governmental standards, including those promulgated by the United States Food and Drug Administration. An acceptable salt of R-salbutamol is for example a hydrochloride, a sulfate, a tartrate, a bromide, a maleate, or a fumarate. More preferred salts of R-salbutamol are the hydrochloride salt, the hydrogen sulfate salt and the tartrate salt. The hydrogen sulfate salts is often called a sulfate salt or a hemi-sulfate salt.

The term “solvate,” where used herein, refers to a solid phase that contains solvent molecules in addition to R-salbutamol molecules in the crystal lattice.

The terms “formulation(s)” and “composition(s)” are herein considered as being synonyms and are used interchangeably.

The term “substantially free from the corresponding isomer” refers to a single isomer, having an enantiomeric excess (ee) of at least 90%. More preferred is an ee 95% and the most preferred ee is ≧98%. In the present case, R-salbutamol is the eutomer and S-salbutamol is the distomer. Combinations of isomers of adrenergic agonists other than approximately 50/50 exist and all such combinations are included in the embodiments disclosed herein.

The term “topical to the eye”, where used in this document, includes administration to the eye and administrations into one or both of the conjunctival sac(s).

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

The term “cyclosporin” as used herein includes naturally occurring fungal metabolites, such as the cyclosporin A, B, C, D and G as well as synthetic and semi-synthetic cyclosporins, such as for example the dihydro- and the iso-cyclosporins. The preferred cyclosporin is cyclosporin A, although mixtures of at least two different cyclosporins may be used.

If not stated to the contrary, all percent (%) concentrations in this document refer to percentage by weight (w/w).

The terms “gel” and “ointment” are used interchangeably in this document.

### Formulations Containing R-Salbutamol

R-salbutamol formulations for ocular administration described herein can be readily processed by standard manufacturing processes, well known to those skilled in the art. The choice of an appropriate method for sterilization is within the scope of understanding of a person of ordinary skill in the art of manufacturing ocular dosage forms. R-salbutamol is readily soluble in water and R-salbutamol is chirally and chemically stable in water solutions. Thus R-salbutamol compositions, which are stable to temperature, can be readily autoclaved after the filling into the final containers.

The embodiments disclosed herein provide pharmaceutical compositions, which comprise R-salbutamol formulated together with selected excipients. The pharmaceutical compositions concern formulations of R-salbutamol that are intended for topical ophthalmic use by patients suffering from dry eye disease.

The lowest tolerated pH of ocular formulations is known to be about pH 4, since formulations with acidity below pH 4 may induce chemical burns (Wright, 2009). The highest tolerated pH may coincide with the normal human tear acidity, which has been found to be 7.0 on an average (Abelson et al. 1981, which publication is hereby incorporated by reference.) Thus, the acidity of ocular formulations of R-salbutamol should be from about pH 4 to about pH 7, preferably from about pH 4.6 to about pH 6.5.

The tonicity of ocular formulations should be isotonic to human lacrimal secretions (Benjamin et al., 1983; and Craig et al., 1995.) or slightly hypotonic. It was therefore determined that the tonicity of R-salbutamol should be adjusted to between 100 mOsm and 1000 mOsm, more preferred between 150 mOsm and 450 mOsm, most preferably between 230 mOsm and 330 mOsm. As used herein, the term “mOsm” is a measurement of osmolality and refers to milliosmoles per kilogram of solvent.

The viscosity of R-salbutamol formulations should be within a range that feels comfortable to the patient, while not causing blurring of the vision. Furthermore, the R-salbutamol formulations should have a viscosity that can be handled easily during manufacturing and filling. It was determined that the R-salbutamol formulations should have viscosity of about 1.0 to about 100,000 centipoise (cP), preferably between about 2.0 to about 90,000 cP and most preferably from about 2.5 to about 75,000 cP, when tested at room temperature. As used herein, the term “cP” indicates a measurement of viscosity and refers to centipoise (water has the viscosity of 1 centipoise at 20° C.).

All compositions intended for use in the eye are required to be sterile. The choice of an appropriate method for sterilization is within the scope of understanding of a person of ordinary skill in the art of manufacturing ocular dosage forms. R-salbutamol compositions, in accordance with the embodiments disclosed herein, which are stable to increased temperatures, can be sterilized by moist heat (autoclaving).

The term autoclaving relates to a standardized thermal heating procedure characterized by: Heating a test composition to 120° C. or more for a period of 15 minutes or more, wherein said composition is aqueous. Said aqueous composition is kept in a closed vessel, which vessel is typically a plastic or glass bottle. The pressure during autoclaving is typically 1 bar or more. The autoclaving may preferably range from 120 to 150° C., more preferably from 120 to 140° C.; the time needed may preferably range from 15 to 120 minutes, more preferably from 15 to 60 minutes; and the pressure applied may preferably range from 1 to 20 bar, more preferably from 1 to 10 bar, and even more preferably form 1 to 5 bar.

Alternatively, ocular R-salbutamol compositions can be exposure to ultraviolet rays or to irradiation, such as gamma irradiation. Formulations can also be processed aseptically, which includes filtration through sterilizing grade filters, which may have a nominal pore size of 0.22 μm.

Maintaining sterility in multiple-use containers is usually achieved by adding one or more preservatives to the formulations. Alternatively, sterilized single-unit dose packages, such as for example single unit dose vials, ampoules, syringes or similar devices, containing a sterile R-salbutamol formulation, as described herein, may be used.

To avoid ocular irritation by foreign particles, all formulations have to be foreign particulate free. The term “foreign particulate free” indicates the absence of any particulate matter, but excludes drug particles, controlled release micro-particulates and the like.

R-salbutamol compositions can be filled into vials, ampoules, syringes or the like and then lyophilized. Lyophilized products, which are free from moisture, are then reconstituted before administration providing a prolonged shelf life of the final product.

### Excipients Compatible with R-Salbutamol

It is known to those skilled in the art of formulating ophthalmic compositions that in order to be accepted to the eye and the tissues surrounding the eye, said composition must have an acidity with a pH from about pH 4 to about pH 7, a viscosity ranging from about 0.5 to about 1000 cps, and an osmolality between about 100 and about 1000 mOsm. Ocular formulations intended for repeat-dose eyedropper devices also must inhibit growth of microorganisms, such as for example bacteria, fungi and molds, while not causing pain, irritation or other side effects to the eye. The challenge is to obtain a composition containing a therapeutically active compound in a specific concentration that meets said criteria, while avoiding incompatibilities with the active pharmaceutical ingredient and simultaneously offering chemical and chiral stability that will translate into a multi-year shelf-life of the formulation over a wide temperature range. A “pharmaceutically acceptable formulation” will meet these criteria and should preferably be autoclavable.

Pharmaceutically Acceptable Ocular Formulations of R-Salbutamol

Numerous ocular excipients have now been investigated in order to determine their compatibility with R-salbutamol. Said excipients are for example antioxidants, buffers, chelating agents, emollients, emulsifiers, fillers, gelling agents, humectants, preservatives, solvents, stabilizers, surfactants, tonicity agents and viscosity modifying agents. It can be noted that one and the same excipient can belong to various classes; thus, for example edetate (EDTA) can have buffering activity, chelating activity, preservative activity, stabilizing activity (also during autoclaving procedures), viscosity modifying activity and possibly additional activities. Another example is propylene glycol that can be used as a solvent, moisturizer and tonicity modifier.

The term “EDTA”, as used herein, comprises the chemical compound ethylenediaminetetraacetic acid and the disodium and calcium disodium salts thereof. EDTA and the salts thereof have many names, such as for example edetate, disodium edetade. ED3A (ethylenediaminetriacetic acid) may be used instead of or in addition to EDTA in the compositions described herein.

Antioxidants are compounds that act to slow or prevent the oxidation of other chemicals. Suitable antioxidants that are compatible with R-salbutamol include sulfites, ascorbates, acetylcystein, butylated hydroxyanisole (BHA) and butylated hydroxytoluene (BHT). When needed, compatible antioxidants can be used in all formulations mentioned herein. Useful concentrations range from about 0.05 percent to about 3 percent, preferably 0.1 percent to 0.25 percent, by weight.

Buffering agents are used to adjust the pH of a solution. The function of a buffering agent is to drive an acidic or alkaline solution to a certain pH range and prevent a change from this pH. Buffering agents have variable properties—some are more soluble than others; some are acidic while others are basic. Suitable buffering agents that are compatible with R-salbutamol include phosphates, boric acid, borates, citrates and acetates. Buffers will be used in the concentrations needed to stabilize the acidity between about pH 4.6 and about pH 6.5. The amount of each of the buffering compounds needed may range from about 0.01 percent to about 4 percent by weight, preferably from 0.05 percent to 1 percent by weight. Ocular compositions with pH≧4.0 or above normal pH of the normal lacrimal secretions are usually not well accepted by patients and ocular compositions of R-salbutamol with pH≧6.5 have now been found to decrease long-term chemical stability. Thus, the acidity of ocular R-salbutamol compositions should be between about pH 4.6 and about pH 6.5.

When needed, compatible buffering agents can be used in all formulations mentioned herein. The acidity of all formulations described herein can be adjusted by changing the concentrations of the buffering agents or by adding an acid or a base as known to those skilled in the art.

Chelating agents, which are often organic compounds, are also called chelants, or sequestering agents and have the ability to form a chelate complex with a substrate. Known chelating agents are for example, edetate, proteins, polysaccharides, polynucleic acids and chelating polymers. Suitable chelating agents compatible with R-salbutamol are edetate and chitosan polysaccharides. If needed, chelating agents may be used in concentrations from about 0.01 percent to about 10 percent, preferably from 0.01 percent to 2.0 percent by weight. Some chelating agents, for example chitosan polysaccharides, also have mucoadhesive properties. When preferred, compatible chelating agents can be used in all formulations mentioned herein.

Emollients can be used in the ocular formulations of the embodiments disclosed herein only if said emollients meet the criteria of being active at pH 6.5 and if they do not decrease the chiral or chemical stability of R-salbutamol. Suitable emollients compatible with R-salbutamol include, for example, glycerin, propylene glycol, and hypromellose (hydroxypropyl methylcellulose, HPMC). When needed, compatible emollients can be used in all formulations mentioned herein. Said emollients can be used in concentrations from about 0.1 percent to about 10 percent and preferably in concentrations from 0.1 percent to 2 percent by weight in the R-salbutamol formulations described herein.

Gelling agents (viscosity-modifying agents) are used to thicken and stabilize liquid solutions, emulsions and suspensions, thereby inducing retention of the compositions in the eye. Gelling agents dissolve in solutions, giving an appearance of a more or less solid matter, while being mostly composed of a liquid. Examples of suitable gelling agents compatible with R-salbutamol include edetate (EDTA), alginic acid and alginates, carrageenan, pectin, gelatin and gelling polymers. When needed, compatible gelling agents can be used in all formulations mentioned herein. Gelling agents can be used in concentrations from about 0.05 percent to about 10 percent and preferably in concentrations from 0.1 to 2.5 percent by weight.

In situ gelling agents may be included in ocular formulations of R-salbutamol and are instilled as drops into the eye and undergo sol-to-gel transition after application to the eye, due, for example, to ion-triggered activation, pH-triggered activation or thermal activation. Examples: Alginate is a gelling agent that can be used in combination with the viscosity-enhancing agent hydroxypropyl methylcellulose (HPMC). The rheological behavior of the alginate/HPMC solutions were retained in the presence of R-salbutamol and may be a useful ion-activated in situ gelling system for R-salbutamol-containing compositions. Polyacrylic acid (Carbopol) is a gelling agent in combination with the viscosity-enhancing agent hydroxypropyl methylcellulose (HPMC) and is a useful pH-triggered in situ gelling system for R-salbutamol-containing compositions. Poloxamer 407 is a polymer with a solution viscosity that increases when its temperature is raised to the eye temperature (Hongyi et al. 2006, Abstract; the disclosure of which is hereby incorporated by reference). The temperature-sensitive rheological behavior of Poloxamer 407 or Poloxamer 407/188 mixtures was not influenced by the presence of R-salbutamol. Suitable in situ gelling agents compatible with R-salbutamol were also found to include alginate/hydroxypropyl methylcellulose, polyacrylic acid/hydroxypropyl methylcellulose. In situ gelling agents as described above can be used in concentrations from about 0.5 percent to about 10 percent, preferably from 0.1 percent to 2.5 percent by weight. Poloxamers can be used in higher concentrations, up to 25 percent by weight.

Humectants can be used to soften biological tissues as they increase the water-holding capacity of ocular tissues, such as the cornea and the conjunctival membranes and certain humectants were found to be compatible with R-salbutamol and can be used in ocular formulations of R-salbutamol. Suitable humectants that are found to be compatible with R-salbutamol include polyethylene glycol, sorbitol and propylene glycol. When needed, compatible humectants can be used in all formulations mentioned herein. Said humectants are used in concentrations from about 0.05 percent to about 10 percent, preferably from 0.1 percent to less than 4 percent and more preferably from 0.1 percent to 2 percent by weight.

Lubricants can hold moisture on the eye. Numerous polymers can be used as ocular lubricants. Suitable lubricants that are compatible with R-salbutamol include methylcellulose, hydroxypropylmethylcellulose, hydroxyethylcellulose, thiolated acrylic acid polymers, carbomer, carboxymethylcellulose sodium, chitosans, and polyisobutylcyanoacrylate. When needed, compatible lubricants can be used in all formulations mentioned herein. If needed, the concentrations of said lubricant is from 0.1 percent to 10 percent, preferably from about 0.1 percent to about 4 percent and more preferably from 0.1 percent to 2 percent by weight.

Mucoadhesive agents refer to materials that will adhere to mucus and mucosal membranes. Suitable mucoadhesives that are compatible with R-salbutamol formulations described here include thiolated acrylic acid polymers, chitosan, polyisobutylcyanoacrylate and ethylcellulose. Mucoadhesive polymers, such as mucoadhesive chitosan and mucoadhesive chitosan-coated microspheres or liposomes will be useful for prolonged delivery of R-salbutamol to the eye. Mucoadhesive agents can be used in concentrations from about 0.1 percent to about 10 percent, preferably from 0.1 to 2 percent by weight. If needed, compatible mucoadhesive agents can be used in all formulations mentioned herein. Using compatible mucoadhesive agents, R-salbutamol can be administered to patients as ocular mucoadhesive minitablets, microspheres and as ocular gel-forming minitablets (see Example 7 below).

Preservatives are substances that can be used to prevent the growth of microorganisms in ophthalmic formulations. Suitable preservatives that are compatible with R-salbutamol include stabilized oxychloro complexes, benzalkonium chloride (BAK), polyhexamethylene biguanide (PHMB) or polyhexamide hydrochloride (HEX). A suitable concentration of a stabilized oxychloro complex is from 0.003 percent to 0.01 percent by weight and a suitable concentration of BAK is from 0.0001 percent (1 ppm) to 0.05 percent (500 ppm) preferably 0.0001 percent to 0.02 percent by weight. A suitable concentration of PHMB is from 0.00001 percent (0.1 ppm) to 0.005 percent (50 ppm), preferably from 0.0005 percent (5 ppm) to 0.00005 percent (0.5 ppm). A suitable concentration of HEX is from about 0.001 percent to about 0.1 percent, preferably from about 0.01 percent to about 0.02 percent. Any preservative mentioned here may be combined with one or more other preservatives for improved efficacy. The concentrations of preservatives may be kept lower than shown here, including the case where no preservatives are used.

In addition to water, which is the preferred carrier, other solvents like polyethylene glycol (PEG) and/or propylene glycol (PG) can be used in ophthalmic compositions. R-salbutamol can be readily dissolved in water in concentrations in excess of five percent by weight. Suitable non-aqueous solvents include polyethylene glycol (about 0.1 percent to about 90 percent) and propylene glycol (about 0.1 percent to about 90 percent). BAK, PHMB and/or HEX can be used in all formulations mentioned herein.

Stabilizers in ophthalmic formulations enhance the physical stability of ocular compositions, such as for example emulsions. It was found that several known stabilizers were not compatible with R-salbutamol since hazy suspensions were formed instantaneously or over time (hours or days). Suitable stabilizers that are compatible with R-salbutamol include methylcellulose, edetate, chitosan, hydroxypropylmethylcellulose and hydroxyethylcellulose. Terms, such as “stabilization”, “stabilizer”, “stability”, when used herein relate to the stability of the pharmaceutical formulation in total and in particular to the stability of R-salbutamol when exposed to storage, oxygen, air, light and/or heat (including high-temperature sterilization, such as autoclavation). The compatible stabilizers listed here are usually used in concentrations from about 0.05 percent to about 4 percent and preferably from 0.05 percent to 2 percent by weight.

Combined stabilizer/solubilizers may be used in formulations containing R-salbutamol. Such combined additional stabilizer/solubilizers are for example cyclodextrins. A preferred cyclodextrin is in particular selected from the group of α-cyclodextrin, β-cyclodextrin, γ-cyclodextrin, hydroxypropyl-β-cyclodextrin, hydroxypropyl-γ-cyclodextrin, dimethyl-β-cyclodextrin and dimethyl-γ-cyclodextrin. The concentrations are generally in the range of from about 0.01 percent to about 90 percent, more preferably in the range of from about 0.1 to about 20 percent by weight.

Surfactants reduce the surface tension of liquids, such as for example water. Suitable surfactants that are compatible with R-salbutamol include nonionic surfactants, such as for example polysorbates, glyceryl stearate, lecithins, polyethoxylated castor oil derivatives and oxyethylated tertiary octylphenol formaldehyde polymers. If needed, compatible surfactants can be used in all formulations mentioned herein. Surfactants are usually used in concentrations from about 0.05 percent to about 4 percent and preferably from 0.1 percent to 2 percent by weight.

Tonicity-adjusting agents increase the effective osmolarity or effective osmolality of a formulation. Hypertonic, hypotonic and isotonic solutions are defined in reference to a cell membrane by comparing the tonicity of the solution with the tonicity within the cell. Ocular compositions preferably contain a tonicity-adjusting agent in an amount sufficient to cause the final composition to have an ophthalmically acceptable osmolality (between 100 mOsm and 1000 mOsm, more preferred between 150 mOsm and 450 mOsm, most preferably between 230 mOsm and 330 mOsm). Suitable tonicity-adjusting agents to be used with R-salbutamol may be of ionic and/or non-ionic type. An example of ionic type tonicity enhancers is sodium chloride and examples of non-ionic tonicity enhancing agents are, for example sorbitol and propylene glycol, which are compatible with R-salbutamol. Thus, R-salbutamol formulations may include for example sodium chloride in concentrations from about 0.1 to about 0.9 percent by weight, sorbitol in concentrations from about 0.1 to about 10 percent or propylene glycol in concentrations from about 0.1 to about 10 percent by weight. If needed, compatible tonicity-adjusting agents can be used in all formulations mentioned herein.

All ophthalmic formulations of R-salbutamol were adjusted to be approximately iso-osmotic to human human lacrimal secretions (Benjamin et al., 1983; Craig et al., 1995.) Over time, increased evaporation leads to increased electrolyte concentration and hyperosmolarity, causing stimulation of expression of metalloproteases, gelatinases, collagenases and stromelysin.

Viscosity-adjusting agents increase the internal friction (“thickness”) of a formulation. The ophthalmic solutions of the embodiments disclosed herein may contain one or more viscosity-adjusting agent and have a viscosity of 1.0 to 100,000 cP, preferably between 2.0 to 90,000 cP, and most preferred between 2.5 and 75,000 cP, which is acceptable since compositions in this range of viscosity feel comfortable to the eye and do not cause blurring of the vision. Viscosity modifying agents can be used in ophthalmic compositions and are substances that have the ability to cause thickening (increase the viscosity) of ophthalmic formulations. Viscosified solutions are accepted to a great degree by patients, mainly because of the ease of administration. Viscosity modifying agents that are compatible with R-salbutamol include edetate, methylcellulose, carboxymethylcellulose, hydroxypropyl methylcellulose, hydroxyethyl cellulose, polyethylene glycol, propylene glycol alginate, chitosan, and tragacanth. The term “hydrogels” is often used for viscosity enhancing excipients, particularly in over-the-counter medications for dry eye disease and refers to a colloid with high gelling ability. If needed, compatible viscosity-adjusting agents can be used in all formulations mentioned herein. When needed, the concentrations of the selected viscosity modifying agents range from about 0.1 percent to about 10 percent by weight, and preferably between 1 percent and 5 percent. Sorbitol may be used as a combined tonicity-adjusting and viscosity-adjusting excipient in a concentration range from about 0.1 to about 10 percent, preferably from 2 percent to 5 percent.

There are currently two strategies to increase the retention time of ocular formulations in the eye: either excipients can be used that have bioadhesive properties, such as for example mucoadhesive excipients, or the formulation can be made more viscous. Both strategies are included in the embodiments disclosed herein.

In certain embodiments, the compositions containing R-salbutamol are packed in opaque plastic containers that may be sterilized using for example ethylene oxide or gamma radiation. A preferred container for an ophthalmic product may be equipped with an eyedropper. Single-dose containers may be used and have advantages that are obvious to those skilled in the art.

### Compositions Compatible with R-Salbutamol

Using excipients that had been found to be compatible with R-salbutamol, compositions such as topical ophthalmic solutions, topical ophthalmic gels, topical hydrophilic ophthalmic ointments, topical ophthalmic emulsions, and topical ophthalmic liposome compositions were prepared. The prepared formulations were tested for physical appearance and stability (refrigerated, room temperature, and at increased temperatures) using standard analytical methodology, well known to those skilled in the art of making ophthalmic formulations.

### EXAMPLES

Certain embodiments are illustrated in the following examples. The embodiments described in this specification are considered to be illustrative in all respects and not restrictive. The scope of the embodiments disclosed herein is indicated by the appended claims, not by this description.

Standard analytical methods, well known to those skilled in the art of analytical chemistry, were used for determination of chiral and chemical stability of R-salbutamol. The excipients used in the present compositions can be analyzed using standard methods that are well known to those skilled in the art.

### Example 1

### Ophthalmic Solutions

Examples of preferred solution formulations containing R-salbutamol sulfate are shown in Tables 1A and 1B.

Preferred solution formulations containing R-salbutamol may contain excipients at different concentrations from those shown in Tables 1A and 1B. Other useful solution formulation containing R-salbutamol may contain excipients that are in part different from those shown in Tables 1A and 1B. The concentration of R-salbutamol can be adjusted up to 10% or even up to 20% and above, while, if necessary, adjusting the concentration(s) of the excipients accordingly.

TABLE 1A
Examples of preferred solution formulations containing
R-salbutamol. (The acronyms refer to the
preservatives used in the formulation)

| Excipients | | | | | |
|---|---|---|---|---|---|
| in % | NOBA [symbol] | LOBAK | MIDBA [symbol] | HIBAK | HEX |
| R-salbu- | 1.0 (1) | 1.0 (1) | 1.0 (1) | 1.0 (1) | 1.0 (1) |
| tamol (%) | | | | | |
| EDTA | 0.100 | 0.100 | 0.100 | 0.100 | 0.100 |
| Boric Acid | 0.095 | 0.095 | 0.095 | 0.095 | 0.095 |
| BAK | — | ≦0.001 | 0.005 | 0.010 | — |
| HEX | — | — | — | — | 0.01 |
| Sorbitol | 4.600 | 4.600 | 4.600 | 4.600 | 4.600 |
| Water | q.s. | q.s. | q.s. | q.s. | q.s. |
| pH (*) | 4.8-6.2 | 4.8-6.2 | 4.8-6.2 | 4.8-6.2 | 4.8-6.2 |

(1) Calculated as free base.
(*) pH is between 4.8 and 6.2, preferably about 6.0. [symbol]
indicates data missing or illegible when filed

TABLE 1B
Examples of preferred solution formulations containing
R-salbutamol. (A large number of formulations were made
and the acronyms refer to R-salbutamol
formulations numbers 6, 7 and 9, respectively)

| Excipients in percent | RSAL6 | RSAL7 | RSAL9 |
|---|---|---|---|
| R-salbutamol (%) | 1.0 (1) | 1.0 (2) | 1.0 (2) |
| Sodium phosphate | 0.473 | — | 0.160 |
| dibasic | | | |
| Sodium phosphate | 0.460 | — | — |
| monobas | | | |
| monohydrate | | | |
| NaCl | 0.480 | — | — |
| BAK (*) | 0.010 | — | 0.010 |
| Sodium citrate | — | 0.300 | — |
| Propylene glycol | — | 1.750 | — |
| Methylparaben | — | 0.030 | — |
| Propylparaben | — | 0.010 | — |
| Methylcellulose | — | — | 0.500 |
| Glycerin | — | — | 2.400 |
| Water | q.s. | q.s. | q.s. |
| pH (**) | 4.8-6.2 | 4.8-6.2 | 4.8-6.2 |

(1) Calculated as free base
(*) The concentrations of BAK may vary between 0.00% and 0.02%.
(**) pH is between 4.8 and 6.2, preferably adjusted to 6.0.

All ophthalmic formulations of R-salbutamol were adjusted to be approximately iso-osmotic to human lacrimal gland secretions (Benjamin et al., 1983; Craig et al., 1995.)

If needed, the tonicity can be adjusted by adding a tonicity-adjusting agent—such as for example saline or propylene glycol—to obtain the preferred tonicity.

If needed, the viscosity can be adjusted by a viscosity-modifying agent—such as for example edetate or hydroxypropyl methylcellulose to obtain the preferred viscosity.

The acidity of the formulations was measured and adjusted by modifying the buffer system or by adding an acid or a base solution to obtain the desired pH.

The solution formulations were prepared by adding the excipients, one at a time to an appropriate amount of water, followed by mixing until dissolved. Once all excipients had been added and dissolved, R-salbutamol was added to the solution of excipients and mixed until dissolved. If needed, viscosity, tonicity and the amount of water were adjusted as indicated above.

### Example 2

### Ophthalmic Ointments and Gels

An example of a preferred composition for a topical hydrophilic ophthalmic gel containing R-salbutamol is shown in Table 2.

Preferred ophthalmic hydrophilic ointments or gels containing R-salbutamol may contain excipients at concentrations that are different from those shown in Table 2. Ophthalmic hydrophilic ointments or gels containing R-salbutamol may contain excipients that are different from those in Table 2.

Topical hydrophilic ophthalmic gel and ointment compositions containing R-salbutamol can keep the drug in the eye for an extended period of time and the prolonged exposure will enhance drug delivery.

Ophthalmic hydrophilic ointments and gels were made, comprising R-salbutamol at concentrations that were usually between 0.1 percent and 5.0 percent, although such formulations may contain in excess of 5.0 percent of R-salbutamol and up to 20 percent and more of R-salbutamol. Said hydrophilic ointment and gel formulations have a viscosity that ranged from 5.000 to 500,000 cP, preferably from 20,000 to 200,000 cP. Examples of thickeners/gelling agents, used in the present studies, are polyethylene glycol 300 and/or polyethylene glycol 3350 and/or polyethylene sorbate (polysorbate) and/or chitosan. A compatible surfactant, such as poloxamer 407 can also be added, preferably in a concentration less than 25 percent, more preferred in a concentration less than 20 percent by weight. It was also found that ophthalmic hydrophilic ointments and gels, containing R-salbutamol, could also contain selected excipients, such as humectants such as for example sorbitol, viscosity modifying agents such as for example methyl cellulose, tonicity agents such as for example NaCl or propylene glycol, chelating agents such as for example edetate or polysaccharides, buffers such as for example phosphate buffers, surfactants such as for example glyceryl stearate, mucoadhesives such as for example polyisobutylcyanoacrylate, antioxidants such as for example BHA or BHT and preservatives such as for example BAK or HEX. Suggested concentrations of these excipients are as shown previously in this document. Said gels and ophthalmic hydrophilic ointments were designed for once-daily ocular administration or for repeated administrations from two to five times daily. The terms “gel” and “ointment” are used interchangeably.

The selected hydrophilic ointment/gel in Table 2 is thick but miscible with water. This composition can hold the drug product in the eye of the patient for an extended time, which will enhance drug delivery.

TABLE 2
Example of a preferred topical hydrophilic ophthalmic ointment or
gel containing R-salbutamol.
(The acronym refers to R-salbutamol gel-formulation #2)

| | Batch RSALGEL2 |
|---|---|
| R-salbutamol sulfate (%) | 1.0 (1) |
| PEG 300 (%) | 69.0 |
| PEG 3350 (%) | 30.0 |

(1) Calculated as free base. The concentration of R-salbutamol can be adjusted up to 10% or even up to 20% while adjusting the concentration(s) of PEG 300 (and/or PEG 3350) accordingly.

Batch RSALGEL2 used a mixture of the polyethylene glycols PEG 300 and PEG 3350 as solvent for R-salbutamol.

The composition of Table 2 was prepared by adding the two polyethylene glycols to a suitable container and heating to 60-65° C. This heating step melts the high molecular weight polyethyleneglycol. Next, R-salbutamol was added and the composition was mixed until the active ingredient was dissolved. Finally, the composition was cooled with mixing to allow the ointment/gel to thicken. The viscosity was 30,000 cP or greater. The pH range for these compositions was not measured since these formulations were non-aqueous. If needed, the tonicity can be adjusted by adding a tonicity-adjusting agent to obtain the preferred tonicity. If needed, any compatible preservative can be added.

### Example 3

### Ophthalmic Hydrophobic Ointments

An example of preferred compositions for topical hydrophobic ophthalmic ointments containing R-salbutamol sulfate is shown in Table 3.

Hydrophobic ophthalmic ointments containing R-salbutamol may contain excipients at concentrations that are different from those in Table 3 and may contain excipients that are different from those shown in Table 3.

The tested hydrophobic ointments were not miscible with water. These compositions can hold the drug product in the eye of the patient for an extended time and will enhance drug delivery.

Ophthalmic hydrophobic ointments and gels may contain R-salbutamol at concentrations between 0.01 percent and 5 percent, more preferably between 0.05 percent and 3 percent, although concentrations of up to 20 percent R-salbutamol can be used. Said ophthalmic hydrophobic ointments and gel solutions were having viscosity in the range of from 5,000 to 500,000 cP and preferably from 20,000 to 200,000 cP. Said ophthalmic hydrophobic ointments and gels have tonicity between 100 mOsm and 1000 mOsm, more preferred between 150 mOsm and 450 mOsm, most preferably between 230 mOsm and 330 mOsm). Said ophthalmic hydrophobic ointments and gels can also contain other excipients, such as for example humectants, viscosity modifying agents, tonicity agents, chelating agents, buffers, surfactants, mucoadhesives, antioxidants and preservatives. Said ophthalmic hydrophobic ointments and gels were designed for once-daily ocular administration or for repeated ocular administrations from two to five times daily to a patient in need thereof.

TABLE 3
An example of preferred hydrophobic ointments (gels) containing R-
salbutamol. As pointed out above, the concentration of R-salbutamol may be
different from 1.0 percent and the concentration of the ointment base (white
petrolatum) and the solvent (propylene glycol) may then have to be adjusted
accordingly.
(The acronym refers to R-salbutamol gel-formulation #4)

| | RSALGEL4 |
|---|---|
| R-salbutamol (%) | 1.0 (1) |
| Propylene glycol (%) | 2.500 |
| Glyceryl stearate (%) | 0.500 |
| Cetyl alcohol (%) | 0.500 |
| White petrolatum | q.s.(2) |

(1) calculated as free base.
(2) quantum sufficit. A preservative can be added

Batch RSALGEL4 contained propylene glycol as a solvent for R-salbutamol, glycerol stearate and cetyl alcohol as surfactants and white petrolatum as base.

The hydrophobic ointment was prepared by dissolving R-salbutamol in propylene glycol. Next, glyceryl stearate, cetyl alcohol, and white petrolatum were added to a suitable container and heated to 65-70° C. This heating step melts the surfactants and the petrolatum. Next, the solution of R-salbutamol was slowly added and the composition mixed until the solvent was dispersed. Finally, the composition was cooled with mixing to allow the ointment to thicken.

If needed, acidity can be adjusted by adding an acid solution or a base solution to obtain the preferred acidity. If needed, tonicity can be adjusted by adding a tonicity-adjusting agent to obtain the preferred tonicity. If needed, viscosity can be adjusted by a viscosity-modifying agent to obtain the preferred viscosity. If needed, a compatible preservative can be added.

### Additional Non-Irritating R-Salbutamol Compositions

To our knowledge, no ocular formulations of R-salbutamol have previously been described. In an embodiment of the present invention it has now been demonstrated that chemically and chirally stable compositions of R-salbutamol can be prepared that do not cause irritation to the ocular tissues (Example 8). The new formulations for R-salbutamol have therapeutic effects in patients suffering from dry eyes, while not causing ocular side effects, such a burning, redness or irritation.

R-salbutamol in the present aqueous formulations has been found to be chirally and chemically stable for at least five years upon storage in a refrigerator or at room temperature (0 to 28° C.).

In certain embodiments, the formulations of the present invention, containing R-salbutamol, deliver therapeutically effective concentrations of R-salbutamol to accessory lacrimal glands and to Meibomian glands after ocular/topical administration of said formulations to the eye or into the conjunctival sac.

Using excipients that have now been found to be compatible with R-salbutamol, compositions such as topical ophthalmic solutions, topical hydrophilic ophthalmic ointments, topical hydrophobic ophthalmic ointments and topical ophthalmic emulsions were prepared and tested. Examples of preferred R-salbutamol compositions useful for patients suffering from xerophthalmia are shown in Tables 1A, 1B, 2 and 3 (above) and in the following tables 4 and 5, where EDTA means ethylenediaminetetraacetic acid (edetate) and BAK means benzalkonium chloride. The preservative compound BAK may be replaced by the preservative compound HEX (polyhexamide hydrochloride) in the concentrations from 0.001% to 0.1% (useful concentration range) or 0.01% to 0.02% (preferred concentration range):

TABLE 4
Examples of preferred solution formulations containing R-salbutamol.
(The acronyms refer to R-salbutamol solution formulations 20, 21 and 10,
respectively)

| Excipients in percent | RSAL20 | RSAL21 | RSAL10 |
|---|---|---|---|
| R-salbutamol (%) | 0.05 (1) | 1.0 (1) | 4.0 (1) |
| EDTA | 0.100 | 0.100 | 0.100 |
| Boric acid | 0.095 | 0.095 | 0.095 |
| BAK (*) | 0.010 | — | 0.010 |
| Sorbitol | 4.6 | 4.6 | 4.6 |
| Water | q.s. | q.s. | q.s. |
| pH (**) | 5.5-6.2 | 5.5-6.2 | 5.5-6.2 |

(1) Calculated as free base.
(*) The concentrations of BAK may vary between 0.001% and 0.02%.
(**) pH is between 4.8 and 6.2, preferably adjusted to 6.0.

Most preferred are compositions of R-salbutamol without any preservative excipient added. Preservative-free formulations of R-salbutamol are particularly useful since the side effects of the preservative agents can then be avoided. To avoid microbial growth, single-dose unit containers of sterile, preservative-free formulations may also be used.

Composition intended for sufferers of xerophthalmia may also contain, as an excipient, hyaluronic acid (MW 750,000 to 2,000,000 daltons) at concentrations from 0.01 percent to 5 percent, which may further improve tear film break up time (Iester et al., 2000, Abstract; Aragona et al., 2002 which publications are hereby included by reference). The term “tear film break up time” as used herein, refers to the time required for the ocular surface to lose cohesive surface wetting after each blink; dry areas will appear as the result of normal evaporation in about 4 seconds and an urge to blink is triggered (Alcon, 2008, which publication is hereby included by reference). R-salbutamol compositions useful for patients suffering from xerophthalmia and containing hyaluronic acid are as shown below, where BAK can be replaced with HEX.

TABLE 5
Examples of R-salbutamol formulations containing hyaluronic acid
(The acronyms refer to R-salbutamol solution formulations
20 and 21, containing hyaluronic acid as dry powder.)

| Excipients | | |
|---|---|---|
| in percent | RSAL20 [symbol] | RSAL21H |
| R-salbutamol (%) (1) | 0.05 (1) | 1.0 (1) |
| Hyaluronic acid | 0.400 | 0.400 |
| EDTA | 0.100 | 0.100 |
| Boric acid | 0.095 | 0.095 |
| BAK (*) | 0.010 | — |
| Sorbitol | 4.6 | 4.6 |
| Water | q.s. | q.s. |
| pH (**) | 5.5-6.2 | 5.5-6.2 |

(1) Calculated as free base.
(*) The concentrations of BAK may vary between 0.001% and 0.02%.
(**) pH is between 4.8 and 6.2, preferably adjusted to 6.0. Concentration of R-salb is between 0.01% and 15%, preferably between 0.05% and 5.0%. Compositions without any preservative agent are most preferred. [symbol]
indicates data missing or illegible when filed

Ophthalmic formulations of low viscosity (<5,000 Cp), which are intended for topical administration to the eye and the surrounding mucous membranes may be applied by means of an eyedropper or a similar device. The volume of each drop depends on the construction of the device, the technique used to produce the drop and the viscosity of the solution being administered. Eyedroppers may deliver from about 30 μL to about 80 μL of the formulation in each drop, preferred is from about 40 μL to about 60 μL of formulation in each drop and most preferred is about 50 μLof the formulation in each drop. Commercial eyedroppers are usually designed to deliver drops with a volume of 50 μL. Thus one drop of R-salbutamol 0.1 percent equals an amount of 50 μg R-salbutamol, calculated as free base. One administration may consist of one to five drops.

A squeezable tube with a small tip is usually used for the administration of gels or ointments to the eye. The amount administered depends on the technique used and the design of the tube. The amount of the gel or ointment dosed is usually from about 10 mg to about 40 mg for each application, although lower (1 to 10 mg) and higher doses (40 to 80 mg) may be administered.

The frequency of administrations solutions, gels, ointments and other formulations can vary from one or less that one administration per week to six administrations daily. More preferred is from one administration to three administrations daily and most preferred is one to two topical administrations daily to the eye.

### Routes of Administration of Compositions with R-Salbutamol

R-salbutamol for ocular indications is preferably administered by instillation to the eye or into the conjunctival sac. Compositions may also be instilled into the nose via nose drops, nasal sprays, or nasal insufflation of dry powder containing R-salbutamol. Alternatively, R-salbutamol may be administered systemically, such as by the oral, intravenous or transdermal routes or by inhalation. Upon systemic administration, the active compound will reach the ocular tissues after systemic absorption and distribution.

Although a beta-2 agonist, such as R-salbutamol, may have ocular therapeutic activity after systemic administration, it is a preferred method to administer drug formulations topically to the eye, for example as solutions, gels, ointments, emulsions, sprays, washes or as topical liposome formulations or as implantable devices that are releasing the beta-2 agonist in a controlled manner. The term “topical to the eye”, as used in this document, includes administration to the eye and administrations into one or both of the conjunctival sac(s). R-salbutamol may also be administered to the eye(s) via devices, such as for example pump-catheter systems, continuous ocular release devices or via contact lenses or ocular minitablets or gel-forming ocular minitablets that contain the active medication. Preferred ocular formulations are solutions, ointments and emulsions.

### Biological Effects of R-Salbutamol in Compositions Thereof

In certain embodiments, both Meibomian and lacrimal gland secretions have now been shown to be increased by administration of formulations containing R-salbutamol (Examples 4, 6 and 7), and it is therefore anticipated that dry eye disease and symptoms thereof will be ameliorated by topical ocular administration of formulations containing R-salbutamol to patients suffering from xerophthalmia. It is also anticipated that the administration of formulations containing R-salbutamol will ameliorate symptoms in patients, who are expected to develop dry eye disease, as previously described herein. Impaired secretion from Meibomian glands is causative in patients suffering from evaporative dry eye (EDE) syndrome. Stimulation of Meibomian gland secretion will therefore have therapeutic value for treating the disease and also for preventing or delaying the onset of the disease in patients at risk for EDE or other types of dry eye disease, such as for example patients with seasonal allergic dry eye disease.

As known to those skilled in ophthalmology, ocular inflammation may, or may in part, be causative to various types of dry eye disease, including EDE. It is also an embodiment of the present disclosure to obtain amelioration of the symptoms of dry eye disorders by the administration of formulations containing a therapeutically effective concentration of an adrenergic beta-receptor agonist, such as for example R-salbutamol, in combination with an anti-inflammatory agent, such as for example a steroid such as for example prednisone, and anti-inflammatory immunosuppressant drug such as for example cyclosporin, a mast cell stabilizer such a for example sodium cromoglycate or a compound with combined anti-inflammatory and antihistaminic activity, such as for example norketotifen or a salt thereof.

### Example 4

### Study on the Affinity of R-Salbutamol for Adrenergic Beta-Receptors

Purpose

The purpose of this study was to investigate the effects of R-salbutamol hemisulfate in several in vitro human β-adrenergic receptor binding assays. The structure of R-salbutamol hemisulfate is shown in FIG. 1.

General Procedure

The affinity for human β₁ receptors was investigated using human recombinant receptors, expressed on HEK-293 cells with [³H](−)CGP 12177, 0.15 nM as the ligand (alprenolol was used as non-specific ligand; 60 min/22° C.) and detection by scintillation counting. The affinity for human β₂ receptors was investigated using human recombinant receptors, expressed on Sf9 cells with [³H](−)CGP 12177, 0.15 nM as the ligand (alprenolol was used as non-specific ligand; 60 min/22° C.) and detection by scintillation counting. The affinity for human β₃ receptors was investigated using human recombinant receptors, expressed on SK-N-MC cells with [¹²⁵I]CYP, 0.6 nM (1 μM (−)propranolol as the ligand ((−)propranolol was used as non-specific ligand; 90 min/37° C.) and detection by scintillation counting. Hill coefficients (n_{H}) were determined by non-linear regression analysis of the competition curves using Hill equation curve fitting. The inhibition constants (Kᵢ) were calculated from the equation Kᵢ=IC₅₀/(1+(L/K_{D}), where L=concentration of radioligand in the assay, and K_{D}=affinity of the radioligand for the receptor).

Results

The specific ligand binding to the receptors is defined as the difference between the total binding and the nonspecific binding determined in the presence of an excess of unlabelled ligand. Results are shown below, where (h) stands for “human”.

Affinity of R-salbutamol to human adrenergic beta-receptors
Summary Results (Crp#9059)

| | | IC₅₀ |
|---|---|---|
| Assay | Test Compound | (M) |
| β₁ (h) | R-SALBUTAMOL.sulfate | 4.5E−0 [symbol] |
| β₂ (h) | R-SALBUTAMOL.sulfate | 8.0E−0 [symbol] |
| β₃ (h) | R-SALBUTAMOL.sulfate | >7.0E− [symbol] |

[symbol]

indicates data missing or illegible when filed

Conclusions. R-salbutamol demonstrated a selectivity of only 5.6 times for adrenergic beta-2 receptors over beta-1 receptors. Thus, R-salbutamol is a relatively non-selective beta-1/beta-2 receptor agonist. R-salbutamol had no affinity for beta-3 receptors.

### Example 5

### Study on the Effects on Lacrimal Gland Secretion after Systemic Administration of R-Salbutamol

Purpose

The purpose of this study was to investigate the effects of R-salbutamol on lacrimal gland secretion after systemic administration of R-salbutamol. It was known that compounds, such as isoprenaline will increase lacrimal gland secretion after intravenous administration (Aberg et al., 1979, Honma U.S. Pat. No. 6,569,903), but R-salbutamol is a vastly different compound from isoprenaline and also from racemic salbutamol, as is well known by those skilled in the art.

General Procedure

Systemic effects of R-salbutamol on lacrimal secretion has now been studied in rabbits, using the rabbit Schirmer methodology described by Aberg et al., 1979. In short, Schirmer strips are strips of filter papers, which are in part inserted into the conjunctival sac and in part hanging out from the conjunctival sac. The Schirmer strips absorb the watery tear fluid as can be observed as wetting of the strips. The length of the wetted area is measured and is an indicator of the amount of available tear fluid in the conjunctival sac. As part of ongoing animal-sparing attempts, only six rabbits eyes were used, which proved to be enough to secure statistically significance.

In the present experiments 30 μg/kg/min of R-salbutamol dissolved in a dose volume of 0.2 ml/kg/min of saline were administered by intravenous (iv) injection over 20 min into a margin ear vein of the conscious rabbits, where “kg” refers to kilograms bodyweight. Basal Schirmer tear flow was measured at 15 to 10 min before, 10 to 5 min before and immediately (5 to 0 min) before the start of the intravenous infusion of the test article. The effects on tear production of 30 μg/kg/min of R-salbutamol were measured at 0 to 5 min, 5 to 10 min, 10 to 15 min and 15 to 20 min after the start of the infusions of R-salbutamol. The average pre-dose value (from three Schirmer readings in each animal) was compared with the average of four readings during the infusions of R-salbutamol. Studies on the effects of R-salbutamol on accessory lacrimal gland tear secretion after topical/ocular administration have also been performed, using Schirmer methodology.

Results

The Schirmer tear secretion values before the start of the intravenous infusions were normal for our laboratory conditions and were stable (approx. 20 mm/5 min) and has previously been found to be unchanged during the intravenous infusion of 0.2 ml/min of saline at room temperature.

When administered intravenously, R-salbutamol 30 μg/kg/min, dissolved in saline, caused the wetting of the Schirmer strips to increase from a predose value of 20.3±1.6 millimeter (average from three measurements in six eyes) to 33.8±2.3 millimeter (average value from four measurements in six eyes) during the intravenous infusion of said concentration of R-salbutamol. The measured effect of R-salbutamol corresponds to an increased wetting of the Schirmer strips of approximately 66 percent. This increase was statistically significant (P<0.001) and indicates a biologically relevant increase in tear fluid volume by R-salbutamol when administered intravenously at a concentration of 30 μg/kg/min (average increase during 20 minutes intravenous infusion).

Conclusion

The systemic (intravenous) administration of R-salbutamol to conscious rabbits caused a significantly increased lacrimal secretion.

### Example 6

### Study on the Effects on Meibomian Gland Secretion after Systemic Administration of R-Salbutamol

Purpose

The purpose of this study was to investigate the effects of intravenous infusion of R-salbutamol hemisulfate on Meibomian gland secretion.

General Procedure

Systemic effects of R-salbutamol on Meibomian gland secretion have now been studied, using Meibometer methodology in the dog. A Meibometer (MB550, Courage-Khazaka GmbH, Koln, Germany) was used to measure the delivery rate of lipids from the Meibomian glands. The measurements were performed in conscious beagle dogs. To reduce variability all studies were performed by a single individual, who had obtained extensive training with the instrumentation and was used to the handling of conscious laboratory dogs. The methodology has been described by Benz, P., et al. 2008, which publication is hereby included by reference, although new and improved computer software was used in the present studies. Meibomian secretion was measured before, during and after intravenous administration of R-salbutamol to conscious dogs. The secretion was expressed as Meibom Units, as expressed by the Meibometer and the values before, during and after a single infusion of 30 μg/kg/min for 20 minutes were recorded and used the calculations of the effects of R-salbutamol on Meibomian gland secretion.

Results

The difference between Meibomian gland secretions before, during and after the infusion of R-salbutamol, were calculated. Pre-infusion secretion was 248±9 MU. During the twenty-minute infusion, the secretion was on an average 235±23 MU and ten minutes post-infusion, the Meibomian secretion was 316±26 MU. Thus, there was an increase of the Meibomian gland secretion after the intravenous infusion time and the difference between pre-infusion and post-infusion secretion was approximately 27 percent. This increase was statistically significant (P<0.05) and indicates a biologically relevant increase in Meibomian gland secretion by R-salbutamol already after a single infusion of R-salbutamol, lasting for only 20 minutes.

Conclusion

A single systemic administration of R-salbutamol to conscious rabbits caused increased lacrimal secretion that was statistically significant and biologically relevant.

### Example 7

### Study on Effects on Meibomian Lipid Secretion after Ocular Instillation of R-Salbutamol

Purpose

The purpose of this study was to investigate effects of ocular instillation of R-salbutamol on Meibomian gland (lipid) secretion.

General Procedure

A Meibometer was used to measure the delivery rate of lipids from the Meibomian glands. The measurements were performed in conscious beagle dogs and the test methodology is described above (Example 6). In these experiments, Meibomian secretion was measured in saline vehicle-treated dogs and in groups of dogs that were treated with a test article, daily at about 9 AM, 11 AM and 1 PM with single eye drops. The two test articles were 4.0 percent R-salbutamol in saline and commercial cyclosporin solution (Restasis®, Allergan). Both eyes were treated and a total of six dogs were used for each treatment group.

Results

The difference between treated and untreated dogs on Day 3 was as follows (mean values; MU=Meibom Units): Vehicle (saline) treated eyes: 174 MU. Eyes treated with R-salbutamol: 304 MU. In the treatment groups, SEM was <10 percent of mean and the difference between the R-salbutamol group and the saline-group was statistically significant (p=0.01). Tests of the reference compound cyclosporin (Restasis®, Allergan) and the saline vehicle did not demonstrate any effect on Meibomian gland secretion after three days of daily applications of cyclosporin. To our knowledge, this is the first demonstration of increased Meibomian lipid secretion in vivo after local ocular administration of any drug and in particular a beta-adrenergic agonist.

Conclusion

To our knowledge, this is the first time increased Meibomian lipid secretion has been demonstrated by local administration of any drug. The adrenergic beta-receptor agonist R-salbutamol increased the Meibomian secretion with statistical significance after three days of ocular/topical applications. There were no effects by Restasis® eyedrops when administered in the same way into the eyes of dogs for three days.

### Example 8

### Test of Ocular Irritation by R-Salbutamol

Purpose

The purpose of this study was to investigate possible side effects of ocular instillation of R-salbutamol, in particular, the risk for development of ocular irritation was studied here.

General Procedure

Nine New Zealand White rabbits were used to evaluate the ocular irritancy of R-salbutamol. As a vehicle control 0.1 ml of 0.9% sodium chloride for injection (B. Braun; Lot No. J9A692) was instilled in the conjunctival sac of the left eye of all the rabbits. Solutions of R-salbutamol in saline were tested at least 48 hours after the conclusion of the placebo/saline tests. Groups of 3 rabbits were administered R-salbutamol in one of the concentration 0.1% R-salbutamol, 0.5% R-salbutamol or 5.0% R-salbutamol. Draize scoring was assessed 30 minutes, 4 hours and 24 hours after the instillations of the placebo control (saline) and the test article solutions.

The grades of ocular reaction (conjunctivae, cornea, and iris) were determined at each examination.

Results

Individual Draize Scoring Sheets were prepared and the mean irritation scores are presented in the following Table.

| | Mean Post-Instillation Irritation Scores | | |
|---|---|---|---|
| Treatment | 30 Minutes | 4 Hours | 24 Hours |
| Vehicle (Saline) | 0 | 0 | 0 |
| 0.1% R-salbutamol | 0 | 0 | 0 |
| 0.5% R-salbutamol | 0 | 0 | 0 |
| 5.0% R-salbutamol | 0 | 0 | 0 |

Conclusions

Based on the results, single-dose R-salbutamol sulfate in saline in concentrations up to 5% were not irritants to the eyes of New Zealand White rabbits.

### Example 9

### Effects on Intraocular Pressure by R-Salbutamol (Study 1)

Purpose

The purpose of this study was to investigate possible side effects of ocular instillation of R-salbutamol on intraocular pressure (IOP) in rabbits with glucose-elevated IOP.

General Procedure

Intraocular pressure (IOP) was increased in conscious rabbits by intravenous injections of glucose. The test compound was administered by instillation into the conjunctival sac immediately after the glucose injection and all measurements were made approximately 12-15 minutes after dosing. R-salbutamol and timolol were tested in three concentrations. Thus, 10 μL of solutions containing 3.5 or 7.0 or 14.0 mg/ml in Tears Naturale® were instilled into the conjunctival sac. Tears Naturale® was used as control solution. TOP was measured using a manometrically calibrated BIO-RAD Digilab pneumatonometer. Tetracaine (10 μl, 0.5%) was applied to the cornea before the IOP measurements. N=7 Dutch Belted rabbits/group (2-4 kg; M and F).

Results

There was no effect on the intraocular pressure by the control solution. As compared with the IOP of the control animals, the intraocular pressure was lowered by timolol by 4.7±1.3 mmHg; 4.6±1.0 mmHg and 4.7±1.3 mmHg for concentrations of 3.5, 7.0 and 14.0 mg/ml of timolol, respectively. IOP was lowered by R-salbutamol by 5.5±1.3 mmHg; 6.7±1.5 mmHg and 5.8±1.3 mmHg for concentrations of 3.5, 7.0 and 14.0 mg/ml of R-salbutamol, respectively. All effects of timolol and of R-salbutamol refer to mean values±SEM and were statistically significant (P<0.05) when compared with the control group. The differences between timolol and R-salbutamol were not statistically significant.

Conclusion

Salbutamol and the reference compound timolol, decreased intraocular pressure in animals with glucose-induced ocular hypertension.

### Example 10

### Effects on Intraocular Pressure by R-Salbutamol (Study 2)

Purpose

The purpose of this study was to investigate possible side effects of ocular instillation of R-salbutamol on “night-time” intraocular pressure (IOP) in rabbits.

General Procedure

Intraocular pressure (IOP) in rabbits is considerably higher during the night than during the day. The present experiments were conducted in Dutch-Belted rabbits in which IOP was higher during daytime as a consequence of switching the night/day cycle by exposing the animals to 12 hours of light during the night and 12 hours of darkness during daytime. The test compounds were instilled into the conjunctival sac at 9.00 AM and IOP was measured at 9.30 AM, 10.00 AM, 1.00 PM, 3.00 PM and 5.00 PM. Three rabbits (6 eyes) were tested for each dose-level and a comparison was made between R-salbutamol and racemic (RS)-salbutamol.

Results

Neither racemic nor isomeric salbutamol increased IOP. Both compounds actually had “normalizing” effects on elevated intra-ocular pressure.

Effects of R-salbutamol and racemic (RS)-salbutamol on “nighttime” intraocular pressures in rabbits.

| Test | Δ mmHg | | | | | | |
|---|---|---|---|---|---|---|---|
| Cmpd | 9:00 | 9:30 | 10 AM | 11 AM | 1 PM | 3 PM | 5 PM |
| R-Lo | 0 | −7 | −9 | −9 | −4 | −3 | −2.5 |
| R-Hi | 0 | −7 | −8 | −11 | −10 | −3 | −2 |
| RS-Lo | 0 | −6 | −6 | −6 | −5 | −2 | −0.5 |
| RS-Hi | 0 | −9 | −9 | −9 | −7 | −4 | −0 |

R-Lo = R-salbutamol 0.33%
R-Hi = R-salbutamol 1.0%
RS-Lo = RS-salbutamol 0.67%
RS-Hi = RS-salbutamol 2.0%.
N = 6 eyes

The time designations are explained above in the section General Procedure

Conclusions

It is concluded that neither salbutamol nor R-salbutamol caused increased intra-ocular pressure.

### Example 11

### Effects on Intraocular Pressure by R-Salbutamol (Study 3)

Purpose

The purpose of this study is to investigate possible side effects of ocular instillation of R-salbutamol on normal intraocular pressure (IOP) in rabbits.

General Procedure

The present experiments were conducted in Dutch-Belted rabbits with normal IOP The test compounds saline-vehicle or R-salbutamol, 4.1% in saline, were instilled into the conjunctival sacs of six rabbit eyes with eye-droppers, twice daily for 5 days. Intraocular pressure was measured twice daily using the methodology stated above.

Results

The effects on IOP were inconsistent and varied from no change from control to a decrease of IOP of 3 mmHg to 5 mmHg. No increase in IOP was observed.

Conclusion

It was concluded that R-salbutamol had minimal or no side effects on normal intraocular pressure in this test.

### Manufacturing of Formulations

The R-salbutamol formulations for ocular administration that are described herein can be readily processed by standard manufacturing processes, which are well known to those skilled in the art. The choice of an appropriate method for sterilization is within the scope of understanding of a person of ordinary skill in the art of manufacture of ocular dosage forms. Thus R-salbutamol compositions, which are stable to temperature, can be readily autoclaved post-processing of the formulation and the filling into the final container.

Ophthalmic carriers are adapted for topical ophthalmic administration, and are for example water, mixtures of water and water-miscible solvents, such as C1- to C7-alkanols, vegetable oils or mineral oils comprising from 0.5 to 5 percent by weight ethyl oleate, hydroxyethylcellulose, carboxymethylcellulose, polyvinylpyrrolidone and other non-toxic water-soluble polymers for ophthalmic uses, such as, for example, cellulose derivatives, such as methylcellulose, alkali metal salts of carboxymethylcellulose, hydroxymethylcellulose, hydroxyethylcellulose, methylhydroxypropylcellulose and hydroxypropylcellulose, acrylates or methacrylates, such as salts of polyacrylic acid or ethyl acrylate, polyacrylamides, natural products, such as gelatin, alginates, pectins, tragacanth, karaya gum, xanthan gum, carrageenan, agar and acacia, starch derivatives, such as starch acetate and hydroxypropyl starch, and also other synthetic products, such as polyvinyl alcohol, polyvinyl pyrrolidone, polyvinyl methyl ether, polyethylene oxide, preferably cross-linked polyacrylic acid, such as neutral Carbopol, or mixtures of those polymers. Preferred carriers are water, cellulose derivatives, such as methylcellulose, salts of carboxymethylcellulose, hydroxymethylcellulose, hydroxyethylcellulose, methylhydroxypropylcellulose and hydroxypropylcellulose, neutral Carbopol, or mixtures thereof. A highly preferred carrier is water. The concentration of the carrier is, for example, from 1 to 100,000 times the concentration of the active ingredient.

### Combinations

Ocular formulations containing an adrenergic beta receptor agonist may contain one or more additional, therapeutically active ingredients. In addition to a beta-receptor agonist, combination compositions may contain therapeutically effective concentrations of one or more other drugs for example belonging to the class of immunosuppressant drugs, such as for example cyclosporin (generic), tacrolimus (Protopic™, Fujisawa) or pimecrolimus (Elidel®, Novartis) or the class of steroids, such as for example fluorometholone, or the class of anti-inflammatory antihistamines, such as for example norketotifen, or the class of antihistamines, such as for example ketotifen and olopatadine, or the class of anti-inflammatory NSAIDs, such as for example bromfenac, or the class of local anesthetics, such as for example bupivacaine, or the class of muscarinic agonists, such as for example pilocarpine.

Some additional examples of compounds belonging to the class of steroids are numerous corticosteroids, such as rimexolone (Vexol®, Alcon), prednisolone acetate (generic), loteprednol etabonate (generic) and difluprednate (Durezol™, Sirion). Some additional examples of compounds belonging to the class of NSAIDS are for example nepafenac (Nevanac™, Alcon), diclofenac (Voltaren™, Novartis), ketorolac (Acular™, Allergan), bromfenac (Xibrom™, Ista), ibuprofen (generic) or indomethacin (generic).

The drugs of the combination therapy, consisting of R-salbutamol and at least one other therapeutically effective compound can be combined in the same composition or can be administered separately, which will make it possible to administer individualized dosing to patients. Using a beta-receptor agonist in combination with another drug may have the advantage of improving the therapeutic activity over single-drug therapy and may also have the advantage of offering lesser toxicity. Thus, for example a combination of R-salbutamol and the significantly more toxic compound cyclosporine, which presently is used for the treatment of dry eyes, will offer a cyclosporin-sparing effect to the patient and will open the possibility to obtain improved therapeutic activity without increasing the doses or the dosing frequency of cyclosporin. Similarly, a steroid-sparing effect may be obtained by the administration of R-salbutamol with the significantly more toxic steroids that may also be used in patients suffering from dry eye syndromes.

All combination products using compositions described herein are included in the embodiments disclosed herein. Thus, although R-salbutamol is preferred, other drugs with affinity for adrenergic beta-receptors may be used instead of R-salbutamol in said compositions and in said combinations.

A preferred combination is a formulation that includes R-salbutamol in a concentration of 0.1 percent to five percent and cyclosporin in a concentration of 0.001 percent to about 1 percent, along with a pharmaceutically acceptable carrier. All combinations are included as embodiments of the present invention.

A useful combination is a formulation that includes R-salbutamol in concentrations from 0.01 percent to 20 percent and cyclosporin in concentrations of 0.001 percent to about 1 percent, along with a pharmaceutically acceptable carrier. Combinations of a beta-receptor agonist and an immunosuppressive drug can be contained in a single formulation or in separate formulations.

Another preferred combination is a formulation containing an adrenergic beta-receptor agonist, such as for example R-salbutamol in a concentration of 0.1 percent to about 5 percent and norketotifen in a concentration of 0.01 percent to about 5 percent, more preferably between about 0.01 percent and about 0.5 percent and most preferred between about 0.02 percent and about 0.4 percent (calculated as base). Said combination formulations containing R-salbutamol and norketotifen have acidity preferably between about pH 4 and about pH 7 and more preferably between from about pH 4.6 to about pH 6.5. The preferred osmolality is between 100 mOsm and 1000 mOsm, more preferred between 150 mOsm and 450 mOsm, most preferably between 230 mOsm and 330 mOsm. The term “norketotifen” as used herein, most often refers to a salt thereof, such as the for example the hydrochloride or the most preferred salt form of norketotifen, which is the hydrogen fumarate salt.

Ocular formulations containing combinations of an adrenergic beta-receptor agonist, preferably R-salbutamol, and an anti-histaminic compound, such as for example ketotifen (Zaditor®, Novartis) are also useful and contain an adrenergic beta-receptor agonist, preferably R-salbutamol in concentrations between about 0.001 percent and about 15 percent (calculated as base), more preferably between about 0.05 percent and about 3 percent (calculated as base) and most preferred between about 0.10 percent and about 2 percent (calculated as base), in combinations with ketotifen in concentrations preferably between about 0.001 percent and about 5 percent, more preferably between about 0.01 percent and about 0.5 percent and most preferred between about 0.02 percent and about 0.1 percent (all percentages are calculated as base). Combinations of a beta-receptor agonist and ketotifen can be contained in a single formulation or in separate formulations.

Ocular formulations containing combinations of an adrenergic beta-receptor agonist, preferably R-salbutamol, and olopatadine contain an adrenergic beta-receptor agonist, particularly salbutamol and preferentially the R-isomer of salbutamol in concentrations between about 0.001 percent and about 15 percent (calculated as base), more preferably between about 0.05 percent and about 3 percent (calculated as base) and most preferred between about 0.10 percent and about 2 percent (calculated as base), in combinations with olopatadine in concentrations preferably between about 0.01 percent and about 2.0 percent, more preferably between about 0.01 percent and about 1.0 percent and most preferred between about 0.02 percent and about 0.4 percent (all percentages are calculated as base). The term “olopatadine” as used herein, most often refers to a salt thereof, such as the for example the hydrochloride salt. Combinations of a beta-receptor agonist and olopatadine can be contained in a single formulation or in separate formulations. Combinations of a beta-receptor agonist and olopatadine can be contained in a single formulation or in separate formulations.

What is claimed is:

1. A method for increasing in vivo Meibomian gland secretion in a mammal suffering from dry eye disease, comprising topically administering to an eye of said mammal a pharmaceutically acceptable ophthalmic formulation containing a therapeutically effective amount of a compound having adrenergic beta-receptor agonistic activity or a salt, solvate or prodrug thereof.

2. The method of claim 1, wherein said compound having adrenergic beta-receptor agonistic activity comprises a compound having adrenergic beta-2 agonistic activity.

3. The method of claim 1, wherein said compound having adrenergic beta-receptor agonistic activity comprises R-salbutamol.

4. The method of claim 3, wherein the concentration of R-salbutamol in said formulation is from about 0.01 percent to about 15 percent.

5. The method of claim 1, where said dry eye disease is evaporative dry eye disease.

6. The method of claim 1, wherein said formulation is administered to the ocular surface of said eye.

7. The method of claim 1, wherein said formulation is administered to the eyelid of said mammal.

8. The method of claim 1, wherein said mammal is a human.

9. The method of claim 1, wherein said mammal is a dog.

10. A method for increasing in vivo Meibomian gland secretion and accessory lacrimal gland secretion in a mammal suffering from dry eye disease, comprising administering to said mammal a pharmaceutically acceptable topical ophthalmic formulation containing a therapeutically effective amount of a compound having adrenergic beta-receptor agonistic activity or a salt, solvate or prodrug thereof.

11. The method of claim 10, wherein said formulation is administered to the ocular surface of said eye.

12. The method of claim 10, wherein said formulation is administered to the eyelid of said mammal.
